(19) United States
(12) Patent Application Publication
(10) Pub. No.: US 2024/0268933 A1
(43) Pub. Date: Aug. 15, 2024

(54) SYRINGE HOUSING FOR ENHANCING FLUID DISPENSING STABILITY

(71) Applicant: ASMPT Singapore Pte. Ltd., Singapore (SG)

(72) Inventors: Kuok Hang MAK, Hong Kong (CN); Shing Chun MAK, Hong Kong (CN); Tsz Kit YU, Hong Kong (CN); Ka Shing KWAN, Singapore (SG)

(21) Appl. No.: 18/107,549

(22) Filed: Feb. 9, 2023

(51) Int. Cl. A61C 9/00; A61M 5/178

(52) U.S. Cl. CPC A61C 9/0026; A61M 5/178; A61J 1/2096

(57) ABSTRACT

A syringe housing has a space that is sized for accommodating a syringe containing a fluid to be dispensed. The syringe is insertable into the syringe housing through an opening at a first end of the syringe housing, and a nozzle configured to engage a dispensing tip of the syringe is located at a second end of the syringe housing opposite to the first end. A syringe adaptor, which includes a pneumatic connector that is connectable to a gas supply for introducing a gas into an air chamber enclosed within the syringe housing and the syringe, is removably coupled to the syringe housing for retaining the syringe after it has been inserted into the syringe housing. The gas is operative in use to apply a force inside the syringe to drive dispensation of the fluid through the nozzle.

### FIELD OF THE INVENTION

This invention relates to the controlled dispensation of fluid in electronic device bonding applications, and in particular, to improving control of an amount of fluid expelled from a syringe containing the fluid during such dispensation.

### BACKGROUND

In the semiconductor industry, adhesive fluid (typically epoxy) used for bonding electronic devices is contained in disposable syringes made of deformable material, such as plastic. Such adhesive fluid in the form of epoxy is dispensed onto a substrate as a means of attachment when bonding semiconductor chips and other relevant devices. Particularly, amongst different methods of dispensing epoxy, a time-pressure dispenser is a type of pneumatic dispenser that allows epoxy to be discharged directly from a plastic syringe. Time-pressure dispensers are user-friendly, as they require no cleaning and they offer plug-and-play operation to minimize set-up time.

However, in order to dispense epoxy, there is a need to connect the time-pressure dispenser to a pneumatic source that supplies air pulses at a regulated pressure to actuate and displace a piston that is located in the syringe. The piston will force the epoxy to be discharged from a nozzle at an opposite end of the syringe. During such pneumatic dispensing, the air pulses cause the plastic syringe to expand significantly in both radial and axial directions due to the low Young's modulus of the plastic material. In many cases, a sealing between the syringe wall and the piston noticeably fails or deteriorates over a number of dispensing cycles, especially when high air pressures are exerted. One reason is that the syringe wall deforms more than the piston at the sealing interface, and this results in a clearance appearing therebetween. Compressed air may then penetrate past the piston through the clearance to be directly introduced as air bubbles into the epoxy. The air bubbles that are trapped in the epoxy cannot escape, except together with the epoxy at the nozzle as the epoxy is being dispensed. Epoxy may also seep through the clearance to accumulate above the piston. Dispensing problems occur because of both the trapped air and the epoxy leak that occur over multiple dispensing cycles.

The use of metallic syringes may help to overcome the above problems, as the high Young's modulus of the metallic material would increase the stiffness of the syringe. However, metallic syringes have not gained popularity due to their high cost and low reusability. Furthermore, syringe cleaning after usage is time-consuming, and even impossible if the epoxy cures in the syringe. In contrast, plastic syringes are more economical and its disposability promotes ease of use.

It would be beneficial to be able to enhance the reliability of plastic syringes by reducing the extent of deformation experienced by the syringes when fluid is being dispensed, and at the same time avoid at least some of the aforesaid shortcomings faced by conventional syringes that are currently in use.

### SUMMARY OF THE INVENTION

It is thus an object of this invention to seek to provide a device for minimizing the expansion of a conventional disposable syringe made of deformable materials during dispensing operations using the syringe.

Accordingly, the invention provides syringe housing for enclosing a syringe containing a fluid to be dispensed, the syringe housing comprising: a space in the syringe housing sized for accommodating the syringe; an opening at a first end of the syringe housing through which the syringe is insertable into the syringe housing; a nozzle located at a second end of the syringe housing opposite to the first end, whereat the nozzle is configured to engage a dispensing tip of the syringe; and a syringe adaptor that is removably coupled to the syringe housing for retaining the syringe after it has been inserted into the syringe housing, the syringe adaptor including a pneumatic connector that is connectable to a gas supply for introducing a gas into an air chamber enclosed within the syringe housing and the syringe, the gas being operative in use to apply a force inside the syringe to drive dispensation of the fluid through the nozzle.

These and other features, aspects, and advantages will become better understood with regard to the description section, appended claims, and accompanying drawings.

### BRIEF DESCRIPTION OF THE DRAWINGS

An embodiment of the present invention will now be described, by way of example only, with reference to the accompanying drawings, in which:

FIG. 1 is a side view of a syringe housing for a syringe according to a first preferred embodiment of the invention;

FIG. 2 is a cross-sectional side view of the syringe housing of FIG. 1 in which a conventional syringe has been inserted;

FIG. 3 is an exploded isometric view of the syringe housing of FIG. 1;

FIG. 4 is a side view of a syringe housing for a syringe according to a second preferred embodiment of the invention;

FIG. 5 is a cross-sectional side view of the syringe housing of FIG. 4 in which a conventional syringe has been inserted; and

FIG. 6 is an exploded isometric view of the syringe housing of FIG. 4.

In the drawings, like parts are denoted by like reference numerals.

### DETAILED DESCRIPTION OF THE PREFERRED EMBODIMENT OF THE INVENTION

During a dispensing cycle, a time-pressure dispenser supplies air pressure to pressurize a syringe to a set pressure for a dispensing process. Inevitably, a plastic syringe would tend to expand when encountering such pressure. The amplitude of the expansion varies with the air pressure applied, and if the pressure is too high, the expansion of the syringe may affect the sealing performance between the walls of the syringe and a piston inside the syringe.

According to two preferred embodiments of the invention as described herein, reduction in the expansion of the syringe is achievable by means of air pressure cancellation and/or by mechanical structure strengthening.

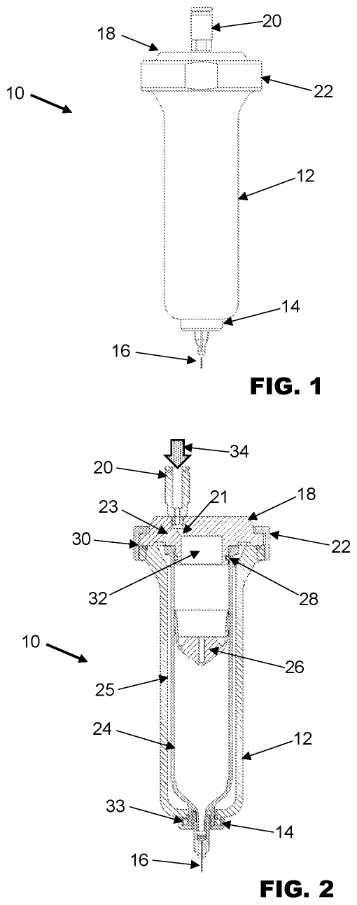

FIG. 1 is a side view of a syringe housing device 10 for a syringe containing a fluid to be dispensed according to a first preferred embodiment of the invention which utilizes the concept of air pressure cancellation. The fluid may be an adhesive fluid such as epoxy. The syringe housing device 10 includes a syringe housing 12 having an interior space that is appropriately sized for inserting and accommodating a syringe, a nozzle 16 located at a tip end of the syringe, and a syringe cap 14 for positioning the syringe relative to the syringe housing 12. The syringe cap 14 may be screwed onto the syringe housing 12 before a syringe is inserted into the syringe housing 12. After the syringe is inserted, the nozzle 16 is configured to engage a dispensing tip of the syringe for dispensing fluid contained in the syringe.

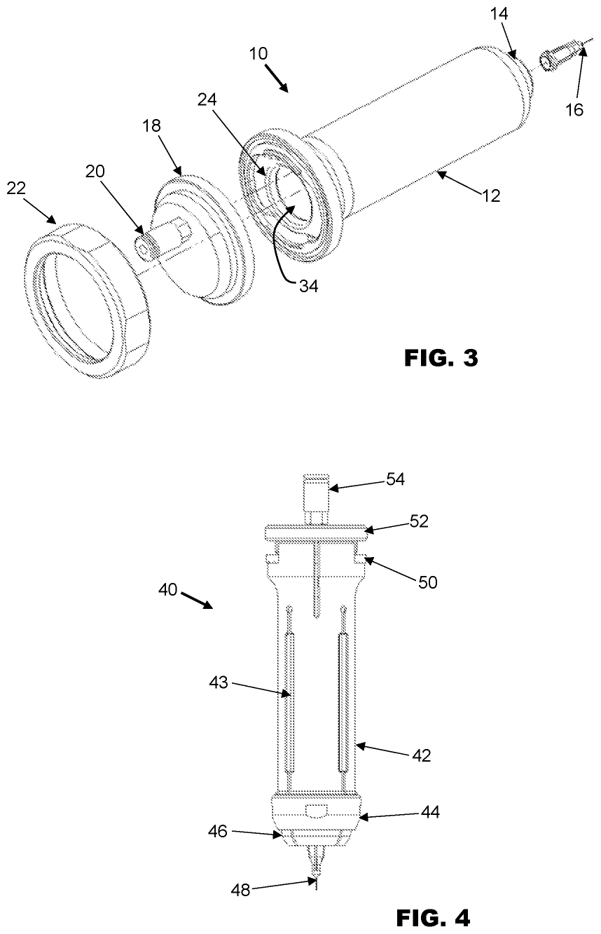

A syringe adaptor 18 is removably coupled to the syringe housing 12 over an opening 34 (see FIG. 3) located at a top end of the syringe housing 12 (that is, opposite to the tip end) in order to connect the syringe to a pneumatic connector 20 after the syringe has been inserted into the syringe housing 12. The syringe is insertable into the syringe housing 10 through the said opening 34. The syringe adaptor 18 is secured onto the syringe housing 12 by an adaptor lock 22 that is designed to be screwed onto the top of the syringe housing 12 to retain the syringe adaptor 18 when it is coupled to the syringe housing 12. Other types of locking mechanisms may also be used to secure the syringe adaptor 18 onto the syringe housing 12. After coupling, the syringe adaptor 18 is operative to retain the syringe that has been inserted into the syringe housing 10.

FIG. 2 is a cross-sectional side view of the syringe housing of FIG. 1 in which a conventional syringe 24 has been inserted. The syringe 24 is typically made from a plastic material. The syringe 24 has a piston 26 that is slidable within the syringe 24 in order to provide a driving force for expelling a fluid contained within the syringe 24 from the tip of the syringe housing 12 through the nozzle 16. A gas, which may comprise air 34 that is supplied in the form of compressed air, is introduced through the pneumatic connector 20 which is connectable to a gas supply of compressed air (not shown) for introducing the air 34 into an air chamber 32 formed between the syringe adaptor 18, the syringe housing 12 and the piston 26. The air chamber 32, being in fluid communication with the piston 26, acquires an air pressure from the introduced air 34 that applies a force inside the syringe 24 to push the piston 26 in the direction of the nozzle 16, thereby driving dispensation of the fluid through the nozzle 16 by expelling the fluid from the syringe 24.

It is notable that according to this embodiment of the invention, an air pressure is further created in a gap 25 between deformable walls of the syringe 24 and walls of the syringe housing 12 after the syringe 24 has been inserted, to prevent the walls of the syringe 24 from deforming excessively when air pressure is received from the introduced air 34 to drive movement of the piston 26. Therefore, while air 34 is introduced into the air chamber 32 via a syringe air inlet 21 formed in the syringe adapter 18, at the same time, the air 34 is simultaneously introduced and received into the gap 25 via a housing air inlet 23 also formed in the syringe adapter 18 into the gap 25 between the syringe 24 and the syringe housing 12. Since the air pressures both in the air chamber 32 inside the syringe 24 and the gap 25 outside the syringe 24 are substantially equalized or equal, the walls of the syringe 24 would be constrained by the air pressure in the gap 25 from expanding or being displaced while the air 34 is being introduced to drive the piston 26 to dispense the adhesive fluid.

For maintaining and equalizing the air pressure both inside and outside the syringe 24, both the air chamber 32 and the gap 25 inside the syringe housing 12 should be hermetically sealed from the ambient atmosphere. As such, a syringe O-ring 28 seals the gap 25 between the syringe 24 and the syringe adaptor 18, an adaptor O-ring 30 seals a gap between the syringe adaptor 18 and the syringe housing 12, and a cap O-ring 33 seals a gap between the syringe housing 12 and the syringe 24.

The objective of such air pressure cancellation is to minimise expansion or displacement due to deformation of the syringe 24 by applying an air pressure to an exterior surface of the syringe 24 to act against any expansion or displacement caused by the air pressure applied for driving the piston 26 to dispense the adhesive fluid. Ideally, the air pressure used to act against the expansion is equal to the air pressure received by the piston 26 for dispensing operations.

When the syringe 24 undergoes the time-pressure dispensing process, the air pressure cancellation allows not only the interior of the syringe to be pressurised, but it also allows the exterior of the syringe to be simultaneously pressurised with an equivalent pressure. Therefore, the pressure acting on the inner wall of the syringe 24 cancels the pressure acting on the outer wall of the syringe 24. As a result, the syringe 24 is constrained from expanding or displacing as a result of the air pressure 34.

As there are small dimensional differences in different brands of syringe 24 on the market, the air pressure cancellation approach is designed to offer some tolerance to accommodate the geometric differences found in different brands of syringes. Beneficially, the syringe housing 12 will not be contaminated by the adhesive fluid as it only contains the syringe 24 but does not contact the adhesive fluid directly, so that the syringe housing 12 can be reused immediately without the need for cleaning.

FIG. 3 is an exploded isometric view of the syringe housing device 10 of FIG. 1. It illustrates how the syringe housing device 10 may be assembled for use. A syringe 24 is first inserted into the syringe housing 12. The nozzle 16 may then be screwed into the syringe housing 12 to contact a tip of the syringe 24, so that fluid that is ejected from the syringe 24 is passed through the nozzle 16 during dispensing. At a top end of the syringe housing 12, the syringe adaptor 18 which includes the pneumatic connector 20 is attached onto the syringe housing 12. An adaptor lock 22 is screwed onto the top of the syringe housing 12 with the syringe adaptor 18 located between the adaptor lock 22 and the syringe housing 12 in order to lock the syringe adaptor 18 against the syringe housing 12. An air hose (not shown) may then be attached to the pneumatic connector 20 for injecting air into the air chamber 32 for driving the piston 26 to eject fluid from the syringe 24.

After the fluid contained in the syringe 24 is depleted, the syringe 24 is removable from the syringe housing 12 for it to be replaced by a new syringe.

FIG. 4 is a side view of a syringe housing device 40 for a syringe 50 according to a second preferred embodiment of the invention which utilizes the concept of mechanical structure strengthening. The syringe housing device 40 includes a syringe housing 42 for inserting a conventional syringe 50 and a nozzle 48 locatable at a tip of the syringe 50. In this embodiment, the syringe housing 42 comprises a flexure arrangement 43 that allows enough space for inserting the syringe 50 when the flexure arrangement 43 is at its unbiased position. After the syringe 24 has been inserted into the syringe housing 42, the flexure arrangement 43 is movable from its unbiased position to a biased position when a flexure lock 44 is screwed onto the flexure arrangement 43 such that flexible fingers of the flexure arrangement 43 terminating at a housing tip 46 of the syringe housing device 40 are arranged to be biased against the syringe 24. This biasing by the flexure arrangement 43 mechanically locks both an axial length of the syringe 24 and cylindrical outer walls of the syringe 24 to provide rigidity to the walls of the syringe 24 after insertion of the syringe 24. The nozzle 48 may also be locked into position by the flexure arrangement 43.

At a top portion of the syringe housing 42, a syringe adaptor 52 is mounted onto the syringe housing 42 in order to connect the syringe 50 to a pneumatic connector 54 after the syringe 50 has been inserted into the syringe housing 42 in use.

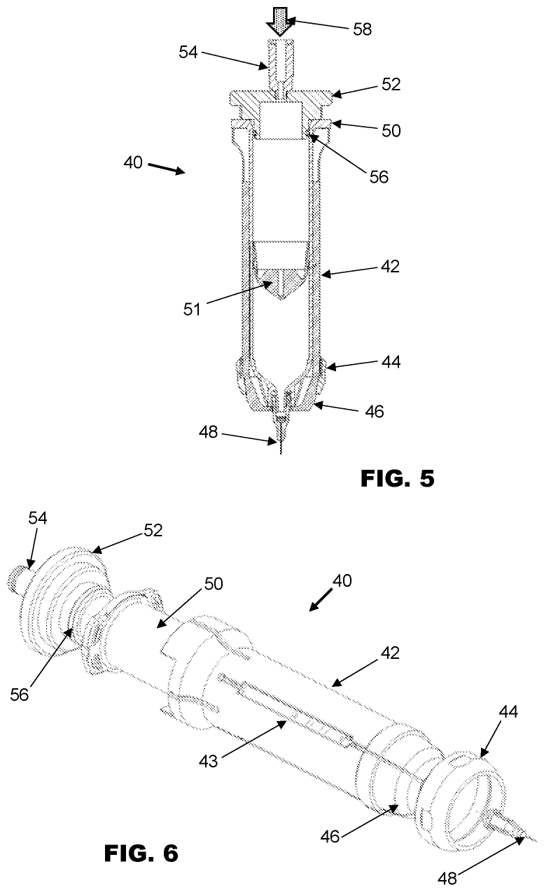

FIG. 5 is a cross-sectional side view of the syringe housing device 40 of FIG. 4 in which a conventional syringe 50 has been inserted. At a tip end of the syringe 50, the nozzle 48 is attached to the syringe 50 and is locked in position by the fingers of the flexure arrangement 43, which have been biased by the action of screwing the flexure lock 44 onto the flexure arrangement 43.

As can be seen in FIG. 5, there is no gap in this embodiment between the syringe housing 42 and the cylindrical walls of the syringe 50. The flexure arrangement 43 acts against deformation of the syringe 50 when pneumatic pressure is introduced against a piston 51 in the syringe 50 during dispensing of fluid from the syringe 50 through the nozzle 48. At the top portion of the syringe housing device 40, the syringe adaptor 52 is pushed into a space in the syringe 50 via interference fit between a syringe O-ring 56 located on an external surface of the syringe adaptor 52 and internal walls of the syringe 50. When air 58 is introduced through the pneumatic connector 54 to drive the piston 51, the syringe O-ring 56 forms a hermetic seal between the internal walls of the syringe 50 and the syringe adaptor 52.

Mechanical structure strengthening is based on the concept of strengthening the structure of the syringe 50 mechanically by inserting the syringe 50 into the syringe housing 42 which is made of a harder material having a higher Young's modulus than that of the syringe 50. The syringe housing 42 is thus designed in a such way that the outer wall of the syringe 50 makes close contact with the inner wall of the syringe housing 42.

In order to facilitate installation, the presence of a flexure mechanism in the form of the flexure arrangement 43 offers a certain degree of radial compliance to enable the insertion of the syringe 50 prior to use. After the syringe 50 has been inserted into the syringe housing 42, the flexure lock 44 is screwed onto and tightened near a distal end of the syringe which pushes the housing tip 46 inwards through a plurality of fingers forming the flexure arrangement 43. These fingers may be in the form of wedge-shaped members, particularly at the housing tip 46. When the flexure lock 44 is fully tightened onto the flexure arrangement 43, the compliance offered by the flexure arrangement 43 is removed and the wedge-shaped members clamp the flexible fingers against the syringe 50. The stiffness of the syringe housing 42 comes into play to reinforce the syringe 50. In this way, the syringe 50 acquires greater resistance to expansion due to air pressure.

FIG. 6 is an exploded isometric view of the syringe housing device 40 of FIG. 4. At a bottom end of the syringe housing 42, the flexure arrangement 43 ends in a housing tip 46, which comprises flexible fingers to clamp onto and secure the nozzle 48 after it has been attached to the syringe 50. The flexure lock 44 is adapted to be screwed onto the flexure arrangement 48 just above the housing tip 46 to bias the flexure arrangement 48 against the syringe 50.

The syringe 50 is insertable into the syringe housing 42 from a top end thereof, after which the syringe adaptor 52 is in turn insertable into the syringe 50, with the syringe O-ring 56 forming an interference fit with the cylindrical walls of the syringe 50. An air hose (not shown) may then be attached to the pneumatic connector 54 for injecting air into the syringe 50 for driving the piston 51 to eject fluid from the syringe 50.

It should be appreciated that the syringe housing devices 10, 40 described with reference to the preferred embodiments of the invention are beneficial for limiting the expansion of a conventional deformable syringe 24, 50 when an adhesive liquid contained in the syringe 24, 50 is dispensed using a time-pressure dispenser.

By restricting the expansion or displacement of the syringe 24, 50, the syringe housing devices 10, 40 ensure reliability of the sealing performance between the walls of the syringe 24, 50 and the piston 26, 51 when dispensing adhesive fluid from the syringe 24, 50. This prevents air 34, 58 from inadvertently being injected into the adhesive fluid and keeps the adhesive fluid from seeping past the piston 26, 51 in an opposite direction. By avoiding these fundamental sealing issues, the dispensing process becomes more consistent and stable.

Although the present invention has been described in considerable detail with reference to certain embodiments, other embodiments are also possible.

Therefore, the spirit and scope of the appended claims should not be limited to the description of the embodiments contained herein.

1. A syringe housing for enclosing a syringe containing a fluid to be dispensed, the syringe housing comprising:
a space in the syringe housing sized for accommodating the syringe;
an opening at a first end of the syringe housing through which the syringe is insertable into the syringe housing;
a nozzle located at a second end of the syringe housing opposite to the first end, whereat the nozzle is configured to engage a dispensing tip of the syringe; and
a syringe adaptor that is removably coupled to the syringe housing for retaining the syringe after it has been inserted into the syringe housing, the syringe adaptor including a pneumatic connector that is connectable to a gas supply for introducing a gas into an air chamber enclosed within the syringe housing and the syringe, the gas being operative in use to apply a force inside the syringe to drive dispensation of the fluid through the nozzle.

2. The syringe housing as claimed in claim 1, further comprising an adaptor lock adapted to be screwed onto the syringe adaptor at the first end of the syringe housing to retain the syringe adaptor when it is coupled to the syringe housing.

3. The syringe housing as claimed in claim 1, wherein the gas comprises compressed air, and the air chamber is located between the syringe adaptor and a piston inside the syringe, the air chamber being configured to acquire an pressure from the compressed air to apply the force against the piston to drive dispensation of the fluid.

4. The syringe housing as claimed in claim 1, wherein the syringe housing is sized to form a gap between deformable walls of the syringe and walls of the syringe housing after the syringe has been inserted, the gap being configured to receive the gas introduced through the pneumatic connector simultaneously with the air chamber.

5. The syringe housing as claimed in claim 4, wherein the syringe adaptor further comprises a syringe air inlet through which the gas is introduced into the air chamber, and a separate housing air inlet through which the gas is introduced into the gap.

6. The syringe housing as claimed in claim 4, wherein a gas pressure in the gap is configured to be substantially equal to a gas pressure in the air chamber, so that deformable walls of the syringe are constrained from expanding by the gas pressure in the gap.

7. The syringe housing as claimed in claim 4, wherein both the air chamber and the gap are hermetically sealed from the ambient atmosphere.

8. The syringe housing as claimed in claim 1, wherein the syringe is configured to be removable from the syringe housing for replacing the syringe.

9. The syringe housing as claimed in claim 1, wherein the housing device comprises a flexure arrangement that is actuable to move between an unbiased position during insertion of the syringe and a biased position to mechanically lock the syringe after it has been inserted.

10. The syringe housing as claimed in claim 9, further comprising a flexure lock that is operative to engage and bias the flexure arrangement to the biased position to mechanically lock the syringe.

11. The syringe housing as claimed in claim 9, wherein the flexure arrangement comprises flexible fingers that terminate at a tip of the syringe housing.

12. The syringe housing as claimed in claim 11, wherein the flexure lock is configured to be screwed onto the flexible fingers for biasing the flexure arrangement.

13. The syringe housing as claimed in claim 12, wherein the flexible fingers are wedge-shaped at the tip of the syringe housing, such that tightening of the flexure lock onto the flexure arrangement clamps the flexible fingers against the syringe for reinforcing the syringe.

14. The syringe housing as claimed in claim 10, wherein the flexure lock is further operative to lock the nozzle at the second end of the syringe housing.

15. The syringe housing as claimed in claim 9, wherein the flexure arrangement is configured to rigidly support the syringe against deformation when the gas is applying a force inside the syringe.

16. The syringe housing as claimed in claim 1, wherein the syringe adaptor is coupled to the syringe housing via interference fit between a syringe O-ring located on an external surface of the syringe adaptor and internal walls of the syringe.

17. The syringe housing as claimed in claim 1, wherein the syringe housing is made of a harder material having a higher Young's modulus than that of the syringe.
